(19) United States
(12) Patent Application Publication
(10) Pub. No.: US 2024/0415833 A1
(43) Pub. Date: Dec. 19, 2024

(54) METHOD OF INJECTING DIHYDROERGOTAMINE INTO THE BODY

(71) Applicant: SUN PHARMACEUTICAL INDUSTRIES LIMITED, Mumbai, Maharashtra (IN)

(72) Inventors: Satyashodhan Babasaheb PATIL, Vadodara (IN); Sudeep Kumar AGRAWAL, Vadodara (IN); Subhas Balaram BHOWMICK, Vadodara (IN); Prashant KANE, Vadodara (IN)

(73) Assignee: SUN PHARMACEUTICAL INDUSTRIES LIMITED, Mumbai, Maharashtra (IN)

(21) Appl. No.: 18/705,130

(22) PCT Filed: Oct. 28, 2022
(86) PCT No.: PCT/IB2022/060399

§ 371 Date: Apr. 26, 2024

Related U.S. Application Data
(63) Continuation of application No. 17/574,816, filed on Jan. 13, 2022.

(30) Foreign Application Priority Data
Oct. 29, 2021 (IN) 202121049646

(51) Int. Cl. A61K 31/4985; A61M 5/00; A61M 5/20; A61M 5/315; A61M 5/32

(52) U.S. Cl. CPC A61K 31/4985; A61M 5/002; A61M 5/20; A61M 5/3158; A61M 5/3202; A61M 5/3243; A61M 2205/581; A61M 2205/584

(57) ABSTRACT

A method is provided for the acute treatment of migraine headaches with or without aura by injecting dihydroergotamine mesylate into a subject. The method includes storing the dihydroergotamine mesylate in an autoinjector at room temperature in a foil pouch. The method further includes removing the autoinjector from the pouch; removing a cap of the autoinjector; pushing the autoinjector down against an injection site of the subject to depress a safety guard; pressing and releasing an activation button on the autoinjector to start injection; and holding the autoinjector against the injection site for at least 10 seconds irrespective of whether a click is generated. The method further includes confirming that the injection is complete by viewing a change of color in a viewing window of the autoinjector, and lifting the autoinjector up away from the injection site and allowing the guard to drop down and lock over a needle.

### CROSS-REFERENCE TO RELATED APPLICATIONS

The present application claims priority to Indian patent application No. 202121049646 filed Oct. 29, 2021, the entire contents of which are incorporated herein by reference.

### FIELD

The present disclosure relates to methods of using auto-injector type devices for injection of ergoline derivatives, such as dihydroergotamine (DHE), ergotamine or pharmaceutically acceptable salts thereof, preferably dihydroergotamine mesylate, and operations of such devices.

### BACKGROUND

Dihydroergotamine (DHE) mesylate is an ergot alkaloid used to treat migraines with or without aura and is a derivative of ergotamine. It is administered as a nasal spray or injection. Dihydroergotamine is ergotamine hydrogenated in the 9,10 position as the mesylate salt. Dihydroergotamine is known chemically as ergotaman-3′,6′,18-trione, 9,10-dihydro-12′-hydroxy-2′-methyl-S′-(phenylmethyl)-, (S′α)-, monomethane-sulfonate. Its molecular weight is about 680 and its empirical formula is C₃₃H₃₇N₅O₅·CH₄O₃S. The chemical structure is:

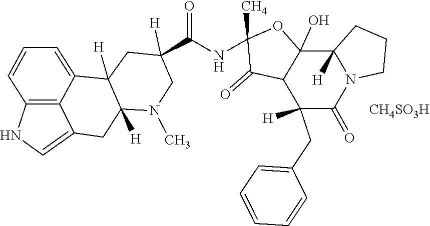

Dihydroergotamine mesylate is indicated in the treatment of migraines and also cluster headaches. For brevity, the following instances of “DHE” refer to dihydroergotamine mesylate, or a pharmaceutically acceptable salt or equivalent thereof.

The dosage and frequency of administration are varied depending upon the indication.

U.S. Pat. No. 6,495,535, which is incorporated herein by reference in its entirety for the compositions disclosed therein, discloses an injectable formulation of dihydroergotamine containing 1 mg/ml or 2 mg/ml of dihydroergotamine mesylate, filled in a medicament container (such as a prefilled syringe). The dihydroergotamine formulation includes dihydroergotamine or salts thereof, a pharmaceutically acceptable liquid vehicle, organic solvents, antioxidants and pH-adjusting agent, with the pH of the dihydroergotamine mesylate composition at 3.6±0.2. The composition according to U.S. Pat. No. 6,495,535 may be provided in a solution having a pH of 3.6±0.2.

U.S. Pat. No. 10,532,049, which is incorporated herein by reference in its entirety for the compositions, chemical structures and formulae disclosed therein, provides a parenteral unit dosage form of dihydroergotamine having reduced isomeric and oxidative impurities as compared to the composition according to U.S. Pat. No. 6,495,535. In particular, U.S. Pat. No. 10,532,049 discloses a parenteral unit dosage form comprising an aqueous solution of dihydroergotamine or a pharmaceutically acceptable salt thereof as a sole active ingredient, one or more pH adjusting agents, and optionally one or more co-solvents, wherein the pH of the aqueous solution is such that it contains a substantially lower amount of impurity of Formula I and a substantially lower amount of impurity of Formula II (shown below) as compared to an aqueous solution having a pH of 4.0 or less, when the parenteral unit dosage form is stored at 25° C. and 60% relative humidity for at least three months.

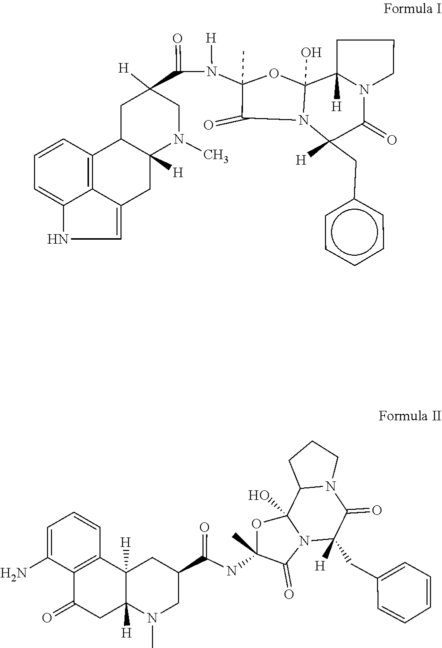

### SUMMARY

The methods described herein provide improved treatment for acute migraine attacks and cluster headache attacks. Individuals undergoing such attacks generally need or desire immediate treatment. The methods described herein provide for such immediate treatment by providing techniques which a patient (who need not be a professional care provider, e.g., a medical professional) can perform himself or herself without visiting a care provider (e.g., a healthcare facility) at the onset of or following an attack.

In at least one embodiment, a method for the acute treatment of migraine headaches with or without aura by injecting ergoline derivatives such as dihydroergotamine, ergotamine, methysergide, or pharmaceutically acceptable salt(s) thereof is provided. Preferably, dihydroergotamine mesylate is injected into a subject in need thereof.

The method includes storing the ergoline derivative such as dihydroergotamine mesylate in an autoinjector at room temperature in a foil pouch; removing the autoinjector from the foil pouch; removing a cap of the autoinjector; pushing the autoinjector down against the injection site of the subject to depress a needle safety guard; pressing and releasing an activation button on the autoinjector to start injection of the ergoline derivative such as dihydroergotamine mesylate into the subject; holding the autoinjector down against the injection site for at least 10 seconds irrespective of whether a click is generated by the autoinjector; confirming that the injection is complete by viewing a change of color in a viewing window of the autoinjector; and lifting the autoinjector up, away from the injection site, and allowing the safety guard to drop down and lock over a needle.

In some embodiments, the method includes generating feedback from the autoinjector when the injection starts. The feedback may be audible, visual, tactile, or any combination thereof. In some embodiments, feedback may be provided multiple times. One or more forms of feedback may be provided at a first time and one or more of the same and/or different forms of feedback may be provided at a later time, for example.

In some embodiments, the method includes generating a click from the autoinjector when the injection starts.

In some embodiments, the aforementioned click is a first click and the method further comprises generating a second click from the autoinjector after the first click and prior to completion of the injection.

In some embodiments, the method further includes activating the activation button after the guard is depressed against the injection site.

In some embodiments, the ergoline derivative such as dihydroergotamine mesylate is injected in a composition without a monoclonal antibody.

In some embodiments, the method is repeated within a 24 hour period, within a one week period, two or more times within a 24 hour period, five or more times within a one week period, or no less than 6 hours after the method is first performed, or any combination thereof. In some embodiments, the method is repeated with a different periodicity.

In some embodiments, the method includes holding the autoinjector down against the injection site for less than 15 seconds.

In some embodiments, about 1 mg of the ergoline derivative such as dihydroergotamine mesylate is injected into the subject.

In some embodiments, less than about 5 mg of the ergoline derivative such as dihydroergotamine mesylate is injected into the subject.

In some embodiments, about 1 mL of a composition comprising ergoline derivative such as dihydroergotamine mesylate is injected into the subject.

In some embodiments, the method includes causing an entirety of a bottommost surface of the safety guard to contact the injection site.

In some embodiments, pushing the autoinjector down against the injection site includes maintaining downward force on the autoinjector throughout injection.

In some embodiments, the method includes viewing the autoinjector, where the autoinjector, including the viewing window thereof, is configured to inhibit light exposure of the dihydroergotamine mesylate stored in the autoinjector.

In some embodiments, when injection begins, a portion of the viewing window changes from a first color to a second color.

In some embodiments, activating the activation button comprises applying less than about 20N of force.

In some embodiments, a method for the acute treatment of migraine headaches with or without aura by injecting an ergoline derivative such as dihydroergotamine mesylate into a subject in need thereof is provided. The method includes removing an autoinjector from a foil pouch, the autoinjector containing the ergoline derivative such as dihydroergotamine mesylate; removing a cap of the autoinjector; pushing the autoinjector down against the injection site of the subject to depress a safety guard, pressing and releasing an activation button on the autoinjector to start injection of ergoline derivative such as dihydroergotamine mesylate into the subject; and holding the autoinjector down against the injection site for at least 10 seconds; and lifting the autoinjector up away from the injection site and allowing the safety guard to drop down and lock over a needle. Completeness of the injection is confirmable by a change of color viewable in a viewing window of the autoinjector.

In some embodiments, a method for acute treatment of cluster headache episodes by injecting an ergoline derivative such as dihydroergotamine mesylate into a subject in need thereof is provided. The method includes storing, in a foil pouch and at room temperature, an autoinjector containing the ergoline derivative such as dihydroergotamine mesylate; removing the autoinjector from the foil pouch; removing a cap of the autoinjector, thereby exposing a safety guard; depressing the autoinjector against a surface of the subject to depress the safety guard; manually actuating the autoinjector to start injection of the ergoline derivative such as dihydroergotamine mesylate into the subject; maintaining the autoinjector in a position in which it is depressed against the surface for at least 10 seconds; confirming the injection by viewing a change of color in a window of the autoinjector; and moving the autoinjector out of contact with the surface and allowing the safety guard to move so as to be situated over a needle.

In some embodiments, actuating the autoinjector comprises actuating a button of the autoinjector before or after the safety guard contacts the surface of the subject.

In some embodiments, the method comprises holding the autoinjector down against the injection site for less than 30 seconds or less than 15 seconds or less than 8 seconds or less than 5 seconds.

In some embodiments, the autoinjector is configured to store no more than 1.5 mL of a composition comprising an ergoline derivative such as dihydroergotamine mesylate.

In some embodiments, the audible feedback is generated when injection starts and later during the injection.

In some embodiments, a method for treatment of cluster headaches by injecting an ergoline derivative such as dihydroergotamine mesylate into a subject in need thereof is provided. The method includes removing an autoinjector containing an ergoline derivative such as dihydroergotamine mesylate from a foil pouch in which the autoinjector is hermetically sealed; removing a cap of the autoinjector, thereby exposing a safety guard; depressing the autoinjector against a surface of the subject to depress the safety guard; manually actuating the autoinjector to start injection of dihydroergotamine mesylate into the subject; maintaining the autoinjector in a position in which it is depressed against the surface for at least 10 seconds, wherein completeness of the injection is confirmable by viewing a change of color in a window of the autoinjector; and moving the autoinjector out of contact with the surface and allowing the safety guard to move so as to be situated over a needle.

In some embodiments, an oxygen scavenger is disposed in the foil pouch.

In some embodiments, the ergoline derivative such as dihydroergotamine mesylate stored in the autoinjector is ready for use when the temperature of the autoinjector containing the ergoline derivative such as dihydroergotamine mesylate is from about 20° C. to about 25° C. or from about 15° C. to about 30° C. In some embodiments, the ergoline derivative such as dihydroergotamine mesylate stored at such temperatures in the autoinjector is ready for use immediately. It is not necessary to refrigerate the autoinjector and wait for a period of time (e.g., 30 minutes) after the autoinjector is removed from the refrigerator before performing the injection. Such aspects allow the autoinjector to be used immediately to combat an acute migraine attack or a cluster headache.

This summary is illustrative only and should not be regarded as limiting.

### BRIEF DESCRIPTION OF THE FIGURES

The disclosed figures depict exemplary techniques of administering DHE and devices for such administration in accordance with some embodiments. In particular, the figures depict elements of a kit for administering DHE and potential locations for injection of DHE on a subject in need thereof.

The disclosure will become more fully understood from the following detailed description, taken in conjunction with the accompanying figures, wherein like reference numerals refer to like elements, in which:

FIG. 1A depicts a kit including an autoinjector according to some embodiments.

FIG. 1B depicts an autoinjector according to some embodiments.

FIG. 2 depicts a cap of an autoinjector according to some embodiments.

FIG. 3 depicts an autoinjector with a cap removed, according to some embodiments.

FIG. 4A depicts a portion of an autoinjector according to some embodiments.

FIG. 4B depicts a first side of a cartridge of an autoinjector according to some embodiments.

FIG. 4C depicts a cross-sectional view of a portion of an autoinjector, according to some embodiments.

FIG. 4D depicts a second side of a cartridge of an autoinjector according to some embodiments.

FIG. 4E is an exploded view depicting a cartridge, a plunger and a sleeve portion of an autoinjector according to some embodiments.

FIG. 5A depicts an autoinjector in a used state according to some embodiments.

FIG. 5B depicts an autoinjector in a used state according to some embodiments.

FIG. 5C depicts an autoinjector in a used state according to some embodiments.

FIG. 6A depicts a kit according to some embodiments.

FIG. 6B depicts an autoinjector in a protective covering according to some embodiments.

FIG. 6C depicts an autoinjector with a viewing window in a first state according to some embodiments.

FIG. 6D depicts an autoinjector with a cap thereof removed according to some embodiments.

FIG. 6E depicts exemplary injection sites.

FIG. 6F depicts an injection orientation according to some embodiments.

FIG. 6G depicts initiation of injection according to some embodiments.

FIG. 6H depicts an injection in progress according to some embodiments.

FIG. 6I depicts an injection in progress according to some embodiments.

FIG. 6J depicts an autoinjector following completion of injection according to some embodiments.

FIG. 6K depicts an autoinjector following completion of injection according to some embodiments.

FIG. 6L depicts disposal of an autoinjector according to some embodiments.

FIG. 7A depicts a partial cross-sectional view of an autoinjector including a stopper.

FIG. 7B depicts a further partial cross-sectional view of the autoinjector shown in FIG. 7A.

### DETAILED DESCRIPTION

Before turning to the figures, which illustrate certain exemplary embodiments in detail, it should be understood that the present disclosure is not limited to the details of methodology set forth in the description or illustrated in the figures. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). It should also be understood that the terminology used herein is for the purpose of description only and should not be regarded as limiting.

As utilized herein, the term “patient” means a user for whom dihydroergotamine is indicated. The term “patient” is used below in describing exemplary operations involving an autoinjector. However, it should be understood that administration of medicament may be carried out by person(s) other than the patient, e.g., a health care provider or assistant, and so references to a particular actor performing operations are not to be construed as precluding performance by other actor(s).

As utilized herein, the term “injection site” means a location on a patient where an injection occurs, e.g., a portion of skin, scalp, exposed tissue, etc.

As used herein, the phrase “unit dosage form” refers to a dosage form meant for single dose administration. The parenteral dosage form is said to be a “unit dosage form” when the aqueous solution filled in the device such as an autoinjector device can be administered to the patient in a single attempt. In certain embodiments, the aqueous solution is free of preservative.

In at least one embodiment, the ergoline derivatives includes ergot alkaloids such as dihydroergotamine, ergotamine and methysergide or pharmaceutically acceptable salts thereof. Preferably, the ergoline derivative is dihydroergotamine mesylate or ergotamine tartrate. More preferably, the ergoline derivative is dihydroergotamine mesylate.

In one embodiment, the method provides for the acute treatment of migraine headaches with or without aura by injecting a composition comprising the ergoline derivative, with other pharmaceutically acceptable excipients such as an alcohol, glycerin with water for injection and acids such as methane sulfonic acid, tartaric acid or a base such as sodium hydroxide, any of the foregoing or any combination of the foregoing. Optionally, the pH may be adjusted to between about 3.4 to about 4.9. Preferably, the composition base a pH in the range of about 4.1 to about 4.9. In some embodiments, the ergoline derivative is administered at a concentration such as a concentration in the range of about 0.5 mg/mL to about 1.5 mg/mL. In some embodiments, the concentration is from about 0.8 mg/mL to about 1.2 mg/mL. Preferably, the ergoline derivative is administered at a concentration of about 1 mg/mL. More preferably, in some embodiments, dihydroergotamine is administered at a concentration of about 1 mg/mL.

As used herein, the percentage of impurities is expressed as percentage by weight of dihydroergotamine.

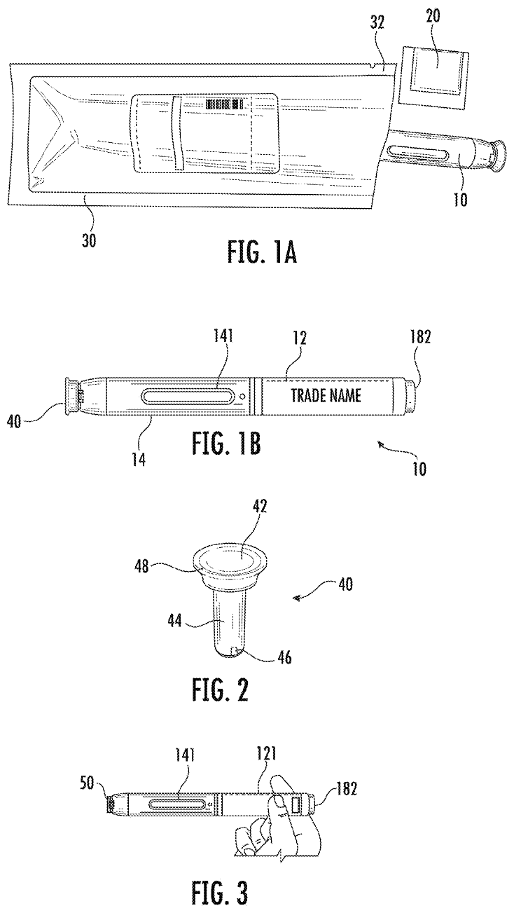

Referring generally to the figures, an autoinjector (such as autoinjector 10 as shown in FIG. 1A, for example, as discussed below) according to one or more embodiments is depicted. With reference to the figures, an exemplary process for administering an ergoline derivative, preferably DHE, using an autoinjector device is described in accordance with at least one embodiment. The ergoline derivative, preferably DHE may be provided in any formulation as described herein. The administration may be self-administration, e.g., when the patient uses the autoinjector to inject the ergoline derivative, preferably DHE, at an injection site of the patient. The figures depict an exemplary device for administering an ergoline derivative such as DHE in accordance with at least one embodiment. General background on the design of injectors is set forth in U.S. Pat. No. 10,617,824 issued Apr. 14, 2020; U.S. Pat. No. 10,159,791, issued Dec. 25, 2018; U.S. Pat. No. 8,986,245, issued Mar. 24, 2015; U.S. Pat. No. 9,022,982 issued May 5, 2015, and U.S. Pat. No. 9,623,182 issued Apr. 18, 2017, the entire contents of each of which are incorporated herein by reference for injector mechanisms.

FIG. 1A depicts a portion of an autoinjector 10 in accordance with at least one embodiment. FIG. 1B depicts an autoinjector 10 prior to use. The autoinjector 10 is provided to a user together with instructions for use. The autoinjector 10 is configured to contain a 1 mL prefilled syringe housing a clear, colorless, sterile solution containing, per 1 mL, 1 mg of an ergoline derivative such as dihydroergotamine mesylate, USP; Alcohol, USP 6.1% by volume; glycerin, USP 15% by weight, water for injection, USP; and methanesulfonic acid and/or sodium hydroxide for pH adjustment, in some embodiments. In some embodiments, less than about 5 mg of DHE is injected into the subject. In some embodiments, the DHE may be injected into the subject in a composition lacking a monoclonal antibody.

The autoinjector 10 is configured to be ready for use at a range of temperatures. Preferably, the autoinjector 10 is stored at room temperature between about 20° C. to about 25° C. (about 68° F. to about 77° F.). The autoinjector 10 may be stored for a brief period (e.g., between about 3 to about 6 hours) at temperatures between about 15° C. to about 30° C. without substantial loss of efficacy. As shown in FIG. 1A, the autoinjector 10 is provided in a foil pouch 30, as discussed in more detail below.

It should be appreciated that FIGS. 1A and 1B, among other figures, depict exemplary dimensions of an autoinjector 10 and that a device having other dimensions may be used in accordance with various embodiments. In some embodiments, the autoinjector 10 is configured to be provided with a needle of 27 gauge and about half an inch in length, although other embodiments may use other needle sizes. In some embodiments, the needle may be 29 gauge and about half an inch in length. The needle extension may be about 5.5 #1.5 mm in some embodiments. In some embodiments, the deliverable volume from the needle may be about 1.0 mL as a minimum volume and about 1.5 mL as a maximum volume. In some embodiments, a pre-filled syringe 16 contained in the autoinjector 10 may have a volume of about 0.1 mL to about 1.2 mL. In some embodiments, the pre-filled syringe may have a volume of about 1.5 mL. The autoinjector 10 may be configured to have about 1.05 mL±2% (about 1.029 ml to about 1.071 mL) of a fillable volume of an ergoline derivative such as DHE stored as a sterile solution. Various embodiments may use different formulations and compositions including those set forth in International Publication WO 2020/044367 and those set forth in U.S. Pat. No. 10,532,049, whose entire contents are referred to herein and incorporated herein by reference for the formulations and compositions, dosages, methods of treatment, and techniques disclosed therein.

Further, it is preferable that the autoinjector 10 is stored in an area away from direct sun. In some embodiments, the autoinjector 10 is provided in a protective covering such as a pouch 30 or overwrap (e.g., a hermetic sealed pouch) made of an opaque material (e.g., foil) designed to prevent light from penetrating from the outside environment to the interior of the pouch. DHE is sensitive to light and to oxygen. Preventing light exposure reduces the potential degradation of light-sensitive DHE (e.g., by reducing the likelihood of impurities).

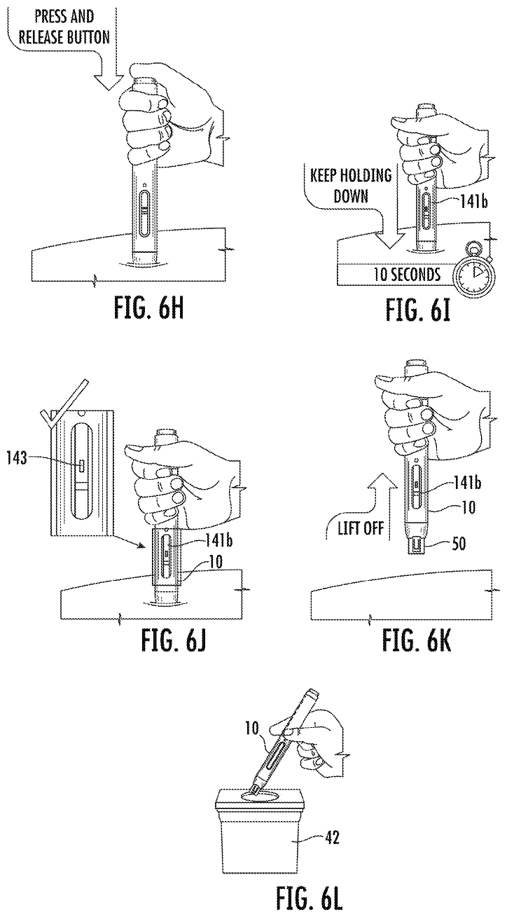

In some embodiments, the autoinjector 10 may be constructed out of durable plastic that inhibits light exposure for the interior contents of the autoinjector 10. The autoinjector 10 is provided and maintained in the pouch 30 until the time of use. The pouch 30 is provided with a frangible portion (a notch, tear-away portion, breakable portion, seam line, etc.) where the pouch 30 may be readily opened with a user's fingers or a tool to access the autoinjector 10. For example, as seen in FIG. 1A, the pouch 30 includes at least one notch 32. In addition to the notch 32, the pouch 30 also includes a seam line 34 as shown in FIG. 6A and FIG. 6B, at which the pouch 30 may be readily tom open to expose its contents. In some embodiments, the pouch may be further provided inside a box or other enclosure that may be substantially impervious to light penetration. The pouch 30 may include a label with information regarding its contents (e.g., drug label information, instructions for use, warnings, or any combination thereof).

The pouch 30 may be provided with one or more oxygen scavengers 20, e.g., in a sachet of about 45 x about 40 mm containing iron powder in activated form and diatomaceous earth, among other components (e.g., electrolyte material, activated carbon and absorbed moisture). The oxygen scavenger 20 may have other dimensions and/or constituents according to various embodiments. For example, in some embodiments, the oxygen scavenger 20 may contain oxygen-absorbing material having an O₂ absorption capacity of about 500 ml. In some embodiments, the oxygen scavenger 20 may be an AGELESS® scavenger as made by Mitsubishi Gas Chemical Company, Inc. of Tokyo, Japan. The sachet may have, as exterior packaging, PET, paper, polyethylene or combinations thereof. In some embodiments, the oxygen scavenger 20 may be configured to indicate a degree of absorption by changing color.

The autoinjector 10) is configured to contain an ergoline derivative such as DHE which may be administered in various amounts and at various periodicities for certain indications. For example, the ergoline derivative such as DHE may be prescribed for administration in response to an acute migraine attack, where a subsequent dose, if necessary, may be administered at least six hours after since a first dose. In some embodiments, up to but no more than six doses or 6 ml are administered in a one week period. In some embodiments, up to but no more than 3 ml or three doses are administered in a day (a 24-hour period). In some embodiments, the autoinjector 10 stores a single dose of a 1 mL volume of liquid medicament including an ergoline derivative such as DHE. Dosing may be carried out subcutaneously in doses of 1 mL.

In some embodiments, a user is guided to check, prior to use, label information on the autoinjector 10 to determine whether the expiration date has elapsed. Furthermore, a visual indication of suitability for use is provided in that the autoinjector 10 is configured to allow at least part of the medicament (an ergoline derivative such as DHE) therein to be visible to a user. Namely, a viewing window 141 (shown in FIG. 1B and discussed below) of the autoinjector 10 may be made out of a partially transparent plastic of sufficient thickness so as to block a portion of light to which the viewing window 141 is exposed, thus serving to protect the contents of the autoinjector 10. If the visible portion of the medicament, which is visible as stored in a glass syringe viewable through the viewing window 141 of the autoinjector 10, appears clouded, has flocculants, particulate matter or other foreign matter, or has an appearance other than clear and colorless, then, in accordance with instructions available to the user, the autoinjector 10 should not be used. One or more air bubbles may be present in the liquid medicament visible through the viewing window 141, and is not an indication of an abnormality or contamination.

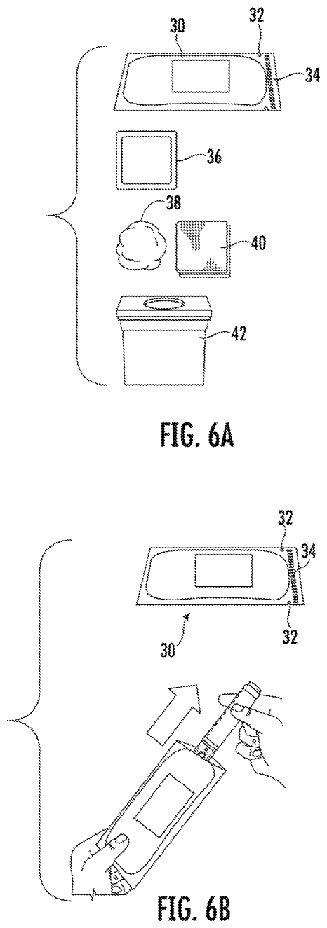

As shown in FIG. 1B, the autoinjector 10 has a body including a first sleeve portion 12 and a second sleeve portion 14. As shown in FIGS. 1B and 4A-4D, an actuator portion 18 is provided proximate an end of the first sleeve portion 12, and a safety cap 40 (which surrounds a needle, as discussed below) is provided proximate an end of the second sleeve portion 14. The sleeve portion 12 may have a label 121 with drug information and/or instructions for use affixed thereon. To use the autoinjector 10, a patient removes the cap 40, which is coupled to an end of the autoinjector body. The cap 40 may be provided in a primary color, such as red, which is readily visible to most users. Further, a color such as red may signify that there is an element requiring due care and caution (e.g., a needle, discussed below).

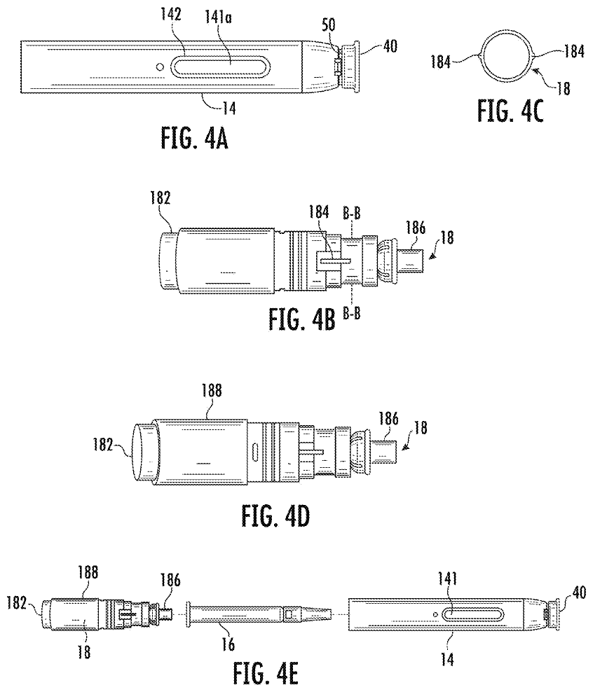

A new and unused autoinjector 10 has its safety cap 40 (a needle cap) attached to an end thereof, as shown, for example, in FIG. 2. In addition, as shown in FIG. 1B, an unused autoinjector 10 has the above-mentioned viewing window 141, which has an appearance of a first color, and an activation button 182. For example, in some embodiments, the autoinjector 10 may have a pink body, a red safety cap 40, a white safety guard 50, a viewing window 141, and a gray activation button 182. In a new, unused autoinjector 10, only part of the safety guard 10 may be visible, as seen in FIG. 1B, which may reduce the likelihood of a user attempting to manipulate this component. Once used, or at least once use has begun, the safety cap 40 is removed from the body and the safety guard 50 containing an injection needle 52 inside is exposed.

The viewing window 141 is between the cap 40 at one end and the activation button 182 at the other end. The viewing window 141 may be configured as an oblong or otherwise elongated aperture formed as a cut-out in a body of the autoinjector 10. Further, the viewing window 141 may be formed by a recessed opening 142 (e.g., an oblong recess in the second sleeve portion 14) of the autoinjector 10. The viewing window 141 is configured such that an appearance of the viewing window changes color during use from a first state 141a in which the contents of the window have a first appearance to a second state 141b in which the contents have a second appearance. In particular, the viewing window 141 permits observation of pre-filled syringe 16 (which should contain clear, colorless medicament before use of autoinjector 10). After use of the autoinjector 10, the viewing window 141 permits viewing of the pre-filled syringe 16 (specifically, a glass barrel thereof) at least partially occupied by the rod portion 186, which may be colored blue, for example. Thus, the second appearance differs in at least one visual characteristic (e.g., color) from the first appearance.

More specifically, during injection, a portion of the autoinjector 10 behind the viewing window 141 (that is, the interior) becomes occupied at least in part by a plunger rod. The plunger rod may be colored (e.g., with a primary color differing from that of the safety cap 40, which may be red, as mentioned earlier, and also differing from the color of the safety guard 50, which may be white; for example, the plunger rod may be blue). Thus, when the plunger rod is positioned so as to be visible (at least in part) through the viewing window, the viewing window 141 has a different color in that the plunger is visible as compared to before use, when the medicament is visible through the viewing window 141. Hence, the change in appearance caused by the physical presence of the plunger viewable in the viewing window 141 serves as a confirmation of completeness of the injection. Thus, the change in color provides instantaneous confirmation of completeness, providing a reassurance to the user that the dose has been administered. Further, as shown in FIG. 6J, a viewing area or viewing slit 143 is provided to allow viewing of a portion of a pre-filled syringe 16 (e.g., a clear barrel of the pre-filled syringe, which may be glass) contained in the autoinjector 10). This allows a user to observe whether the medicament is clear and colorless or in a different state indicating potential contamination.

In some embodiments, the first state 141a and the second state 141b are states where substantially all of the viewing window 141 appears to have a single color (except for minute features). For example, the first state 141a corresponds to the window 141 appearing entirely clear, or substantially so, whereas the second state 141b corresponds to the window 141 appearing entirely or substantially colored.

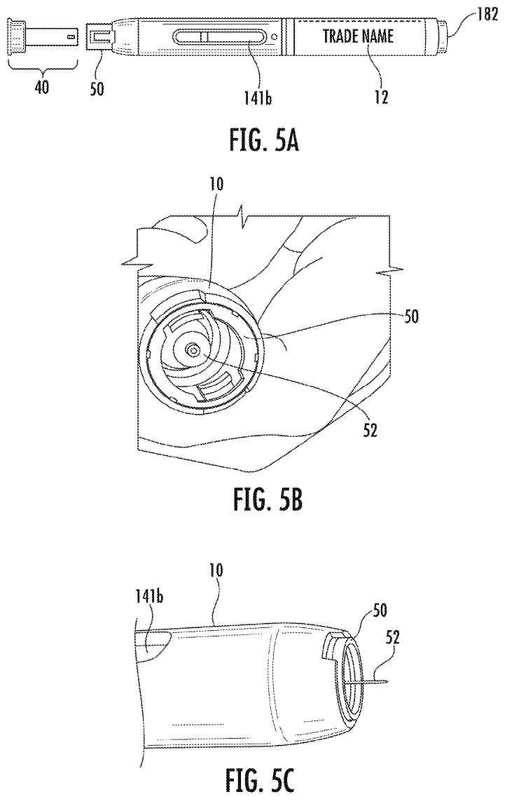

In some embodiments, the user does not remove the cap 40 until the user is ready to deliver the medicament (an ergoline derive such as DHE). To facilitate removal of the cap 40, the cap 40 may be provided with a grip surface element (e.g., a flat knob 42 with a beveled rim 48 that is readily grasped) to which force may be applied by the patient's finger(s) while gripping the cap to dislodge the cap from the body. The knob 42 may be affixed to a post 44 having divots 46 which engage a safety guard 50, shown in FIG. 3. The cap 40 may be provided with a rounded profile defined by rim 48, which is configured to be contacted by a patient's finger(s) when the patient is removing the cap 40 from the body of optionally returning the cap 40 in place. The cap 40 is configured to be pulled off the body in a linear pulling motion, without twisting or turning of the cap 40. The user is instructed to dispose of the cap 40 following removal.

An exemplary process for administering an ergoline derivative such as DHE is now described with reference to FIGS. SA-5C and 6A-6L. In some embodiments, the user is instructed to use the autoinjector 10 in a kit shown in FIG. 6A including the pouch 30 in which the autoinjector 10 is provided, an alcohol swab 36, a cotton ball 38 or gauze 40, and a container 42 for receiving the used autoinjector needle 52 and/or the autoinjector 10 itself. The pouch 30 may be provided in a carton together with instructions for use.

In some embodiments, a user first removes the foil pouch 30 from any exterior packaging (e.g., a box, enclosure or carton) if present. Then, the pouch 30 is opened, as shown in FIG. 6B, and the autoinjector 10 is removed. The removed autoinjector 10 is shown in its unused state in FIG. 6C. Upon inspection of the unused autoinjector 10) (e.g., to check that the viewing window 141 does not appear to show contamination, cloudiness, etc.), the safety cap 40 is removed from one end of the autoinjector 10 so as to separate the safety cap 40 from a body of the autoinjector, as shown in FIG. 6D FIG. 3 depicts the autoinjector 10 with the cap 40 removed. An activation button 182, discussed below, is at an opposite end of the autoinjector 10 from the safety cap 40.

Once the cap 40 is removed from the body, a safety guard 50 at one end of the autoinjector 10 is exposed, as shown in FIG. 6D. The safety guard 50 may be a surrounding member encircling at least part of an autoinjector needle 52 or an entirety of the needle 52 and is configured to inhibit contact between a user's fingers and the needle 52 itself. The user is instructed not to touch the safety guard 50 so as to reduce the likelihood of contact with the needle 52. The likelihood of such contact is lowered because the needle 52 is disposed so as to be inside of, and shielded by, the safety guard 50. The safety guard 50 serves as a needle cover because it is positioned at least over part of the needle 52. The safety guard 50 is configured to be movable between a retracted position and a deployed position. The retracted position is one in which the safety guard 50 is disposed away from the injection site and is withdrawn at least partly into the body of the autoinjector 10. In contrast, the deployed position is one in which the safety guard 50 extends from the body of the autoinjector 10 (see FIG. 6K) and circumferentially surrounds at least part of the needle 52 so as to prevent unwanted needle contact.

In some embodiments, before or after the autoinjector 10 is removed from the pouch 30, the user selects an injection site, e.g., the skin of the middle thigh, as shown in FIG. 6B. In some instances where multiple doses are prescribed, a different site is selected. The user is instructed not to inject into moles, scars, birthmarks or areas of tenderness, bruising, redness or hardness, and not to inject through clothing. The user is instructed not to inject into the same injection site for consecutive injections, and to select an injection site that is at least two inches from an immediate prior injection. The user is instructed to clean the injection site (e.g., with an alcohol swab) prior to injection.

Exemplary injection sites may differ according to various embodiments. Where the injection site is the abdomen, the injection site may be at least two inches away from the patient's navel. When the injection site is the patient's thigh(s), the injection site may be the front, middle portion of the thigh(s). When the patient is not self-administering but instead receiving assistance (e.g., through a health care provider or layperson assisting the patient), the injection site may be the outer area of the upper arms (e.g., the back side of the upper arms), for example.

In some embodiments, the injection may be administered to one or more of a patient's abdomen, thighs, or the dorsal or outer areas of the upper anns. The method of administering an ergoline derivative such as DHE may include rotating the injection site so that the same site is not used for consecutive injections. The method may include spacing a subsequent injection site at least two inches from an immediately previous injection site. Once the injection site is selected, the site may then be prepared for injection, e.g., by cleaning with the alcohol swab 36 and allowing the site to dry before injection.

In some embodiments, the user is instructed to position the needle 52 directly above the cleaned injection site with the safety guard 50 resting on the skin, such that an entirety of the bottommost surface of the safety guard 50 contacts the injection site. The user is instructed to allow the safety guard 50 to be substantially flush against the skin. The needle cover or safety guard 50 may be positioned at about a ninety degree angle relative to a skin surface (this positions the needle 52 at approximately a ninety degree angle relative to the skin surface), as shown in FIG. 6F. Once so positioned, the user then pushes the autoinjector 10 down against the skin to depress the safety guard 50, as shown in FIG. 60. In response to the pressure of the user's skin at the injection site against an end of the autoinjector 10, the safety guard 50 moves to the retracted position so as to be depressed and moved away from the skin. The user may be instructed to keep downward pressure, and, to the extent possible, uniform pressure on the skin (the injection site) for the duration of the injection.

Once the safety guard 50 is depressed against the skin, the user may commence injection by manually actuating (e.g., by pressing, engaging, depressing) the activation button 182 of the autoinjector 10 at the opposite end of the autoinjector body from the safety guard 50. The activation button 182, as shown in FIG. 4A, is part of an actuator portion 18 of the autoinjector 10. The actuator portion 18 includes the activation button 182, an actuator body portion 188, and a terminus that includes a rod portion 186. In some embodiments, the rod portion 186 is a plunger rod of a pre-filled syringe 16 filled with medicament. Specifically, the rod portion 186 is disposed in an interior of a barrel of the pre-filled syringe 16 when the pre-filled syringe is assembled with the actuator portion 18 and the second sleeve portion 14 respectively. The pre-filled syringe 16 is arranged to be held in place by the second sleeve portion 14, and in particular, a syringe carrier of the second sleeve portion 14.

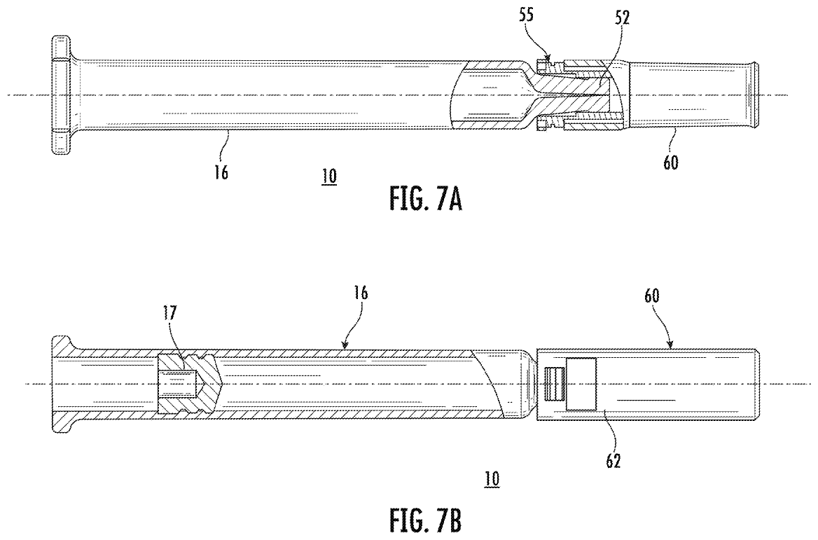

FIGS. 7A and 7B depict partial cross-sectional views of a component of an autoinjector. The exemplary structures shown in FIGS. 7A-7B may be implemented in an autoinjector or portion thereof as shown, for example, in FIG. 4E. As seen in FIG. 7A, needle 52 is disposed to be shielded at least in part by an internal needle cover or needle shield 55 surrounding at least part of the needle 52 circumferentially. The needle cover 55 is structured with a skirt portion at one end thereof that may interface or contact with at least another component of the autoinjector 10. The needle cover 55 may be structured to deform (e.g., to slightly flex inward toward the pre-filled syringe 16 containing needle 52) when pressure is applied. The needle cover 55 may be disposed to be surrounded by a shield 60 that further protects the needle 52. The shield 60 is comparatively greater in rigidity than the needle cover 55. The shield 60 may be provided with an external cap or sheath 62 covering the needle cover 60. The autoinjector 10 is further provided with a stopper 17 shown in FIG. 7B. The stopper 17 is configured to be pushed forward by rod portion 186 to cause medicament to be dispensed from the pre-filled syringe 16.

In some embodiments, the actuator portion 18 is configured with physical features, including the rod portion 186, that interface with the pre-filled syringe 16 of the autoinjector 10 to cause the pre-filled syringe 16 to deliver medicament through the needle 52 of the autoinjector 10. For example, as seen in FIG. 4B, the rod portion 186 is configured for insertion into an end of plunger 16. Further, the actuator portion 18 is configured with protrusions 184 which are shown in FIG. 4C in a cross-section taken at line B-B in FIG. 4B. FIG. 4D shows the actuator portion 18 in an alternative view. As seen in FIG. 4D, the activation button 182 of the actuator portion 18 is configured as a rounded component which is readily manipulated, e.g., by a thumb or finger of a user. The autoinjector 10 may be spring-actuated, i.e., it may utilize a spring actuation mechanism (e.g., a spring, such as a pre-loaded spring, or a concentric arrangement of springs) in the body of the autoinjector 10 to cause the autoinjector 10 to deliver the ergoline derive such as DHE to the injection site. The spring may be configured for acting on the pre-filled syringe 16 to cause a stopper (such as stopper 17 shown in FIG. 7B and discussed above) to be displaced in the body of the autoinjector 10.

The pre-filled syringe 16 is configured to be movable within the second sleeve portion 14 of the autoinjector 10, as shown in FIG. 4E. In some embodiments, however, the user may first actuate the activation button 182 and may then depress the safety guard 50 against the skin. In some embodiments, activating the activation button 182 comprises applying less than about 15N to less than about 25N of force, e.g., less than about 20N of force. Such downward force may be maintained throughout the duration of the injection.

In some embodiments, (1) the safety guard 50 may be pushed against the injection site first without pressing the activation button 182, and then the activation button 182 is pressed, or (2) the activation button 182 is first pressed and then the safety guard 50 is pushed against the injection site to activate the injection. That is, the autoinjector 10) may be actuated by manipulating the activation button 182 of the autoinjector 10 before or after the safety guard 50 contacts the injection site (a surface of a user). In circumstance (1), little or no movement occurs with respect to the activation button 182 or other components, whereas in circumstance (2), the autoinjector components have a degree of play (travel) such that the safety guard 50 may move slightly. In alternative embodiments, the autoinjector 10 is configured to inhibit premature injection by prohibiting injection until the user both activates the activation button 182 and presses the autoinjector 10 against the injection site. In some embodiments, pressing the safety guard 50 against an injection site releases an internal mechanism of the autoinjector 10 from being held by projections of a component which keeps it in a position to trigger the spring for extension. In a similar way, pushing the activation button 182 also releases a spring (e.g., a spring as described above, where the autoinjector 10 is spring-actuated) to thereby trigger or activate the autoinjector 10. In some embodiments, either pressing of the safety guard 50 or depressing of the activation button 182 may take place. In such embodiments, there is no restriction as to which of these actions (pressing the safety guard 50 or the activation button 182) may take place first, as each action is independent. However, in some embodiments, both actions must be performed to fully activate the autoinjector 10.

In some embodiments, once the activation button 182 is activated, a first feedback is generated, e.g., audible feedback in the form of a click, within about 1 to about 3 seconds after injection starts, as indicated in FIG. 6H. The audible feedback serves as an indication that injection has commenced and further indicates that an injection is not yet completed. The user is instructed to maintain the autoinjector 10 in place, against the injection site, for at least about ten seconds, as indicated in FIG. 6I. That is, the autoinjector 10 is maintained in position so as to be depressed against the injection site surface for at least about ten seconds. In some embodiments, the user may hold the autoinjector 10 in place against the injection site for more than about ten and less than about fifteen seconds, or at least about fifteen seconds and less than about thirty seconds.

In some embodiments, second audible feedback (a second feedback output that is audible) may be generated, such as a second click, after the first feedback and prior to completion of injection, as indicated in FIG. 6I. For example, the second audible feedback may be generated between about 1 and about 3 seconds prior to completion of injection. The user is instructed that even if such feedback is emitted before ten seconds have elapsed since the start of the injection, the user should continue to maintain the autoinjector 10 depressed against the injection site for at least ten seconds to ensure that the injection is complete. In some embodiments, the total dose may be delivered in between about three to about four seconds following initiation of injection; however, the user is instructed to maintain the autoinjector 10 in place for about 10 seconds so as to ensure that the delivery is complete. In some embodiments, at least a three second interval is present between audible feedback outputs to the user.

In some embodiments, once the injection is complete, the viewing window 141 changes in color, as mentioned above, due to the viewing window 141 being occupied by at least part of a plunger rod of the autoinjector 10. As injection occurs, the plunger rod moves relative to the viewing window 141 such that the plunger rod occupies a greater extent of the viewing window from the start of injection (where the viewing window 141 is unoccupied by the plunger rod). In some embodiments, the plunger rod position changes relative to the viewing window 141 such that the viewing window 141 gradually becomes filled by the plunger rod over the course of the injection.

Thus, owing to the gradual way in which the plunger occupies the viewing window 141, the user is provided with an indication of the progress of the drug delivery, namely, that the delivery is in progress and not yet complete. In some embodiments, when the injection is complete, the plunger rod occupies all of the viewing window 141, as shown in FIG. 6I. This serves as a visual indication or confirmation of completion of injection. In some embodiments, the combination of the multiple audible feedback outputs to the user and the change in window 141 color (as a form of visual feedback) provide reassurance to the user that the injection has successfully completed. This reassurance is particularly important in connection with the treatment of acute migraines and cluster headaches, since the user is typically the patient himself or herself, due to the importance of obtaining treatment immediately at the onset of an attack.

In some embodiments, the user is instructed to then lift the autoinjector 10 straight up (directly parallel to the direction of injection) away from the injection site, as shown in FIG. 6K. The act of lifting the autoinjector 10—e.g., in a direction opposite to that in which the autoinjector 10) was depressed against the skin—from the injection site causes the safety guard 50 to drop down (e.g., from a retracted position to a deployed position) and lock over the needle 52. Thus, when the safety guard 50 is deployed, the autoinjector 10 is in a “lock-out” state in which the safety guard 50 is locked in place, encircling the needle 52 so as to reduce unwanted contact or interaction with the needle 52.

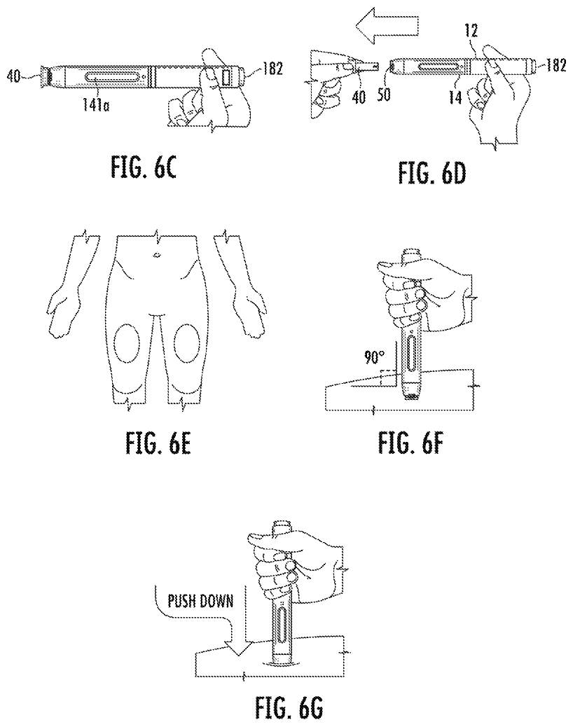

An example of a safety guard “lock-out” state—with the safety guard 50 making up a needle cover extending over the needle 52 and the viewing window 141 being occupied by the plunger rod—is shown in FIG. 5A. The locking of the safety guard 50 over the needle 52 prevents further injections with the needle 52, thus lowering the risk of contamination or injury. In some circumstances, the safety guard 50 may not fully extend past the entirety of the needle 52; that is, the needle 52 may be in a position where a portion thereof protrudes beyond an extent of the safety guard 50 following removal from the injection site, as shown in FIG. 5C. Nevertheless, even in this circumstance, the body of the autoinjector 10 may still be gripped from its sides without contacting the needle 52, as seen in FIG. 5B. The autoinjector 10 may then be disposed of in a waste receptacle (e.g., a “sharps” container 42 shown in FIG. 6L configured to receive sharp items such as needles).

In some embodiments, a method of providing DHE to a subject in accordance with the techniques above may be repeated. For example, following a first injection according to the foregoing operations, one or more subsequent injections may be repeated within a given time period following the first injection. For example, the method may be repeated within a 24 hour period, within a one week period, two or more times within a 24 hour period, five or more times within a one week period, or no less than 6 hours after the method is first performed, or any combination thereof. In some embodiments, the method is repeated with a different periodicity.

As used herein and in the appended claims, singular articles such as “a” and “an” and “the” and similar references in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

The embodiments illustratively described herein may suitably be practiced in the absence of any element or elements, or limitation or limitations. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified. The expression “comprising” means “including, but not limited to.” Thus, other non-mentioned substances, additives, carriers, or steps may be present. Unless otherwise specified, “a” or “an” means one or more.

Unless otherwise indicated, all numbers expressing quantities of properties, parameters, conditions, and so forth, used in the specification and claims are to be understood as being modified in all instances by the term “about” or “approximately.” Accordingly, unless indicated to the contrary, the numerical parameters set forth in the following specification and attached claims are approximations. Any numerical parameter should at least be construed in light of the number of reported significant digits and by applying ordinary rounding techniques. The terms “about” or “approximately,” or “substantially,” or similar terms, when used before a numerical designation. e.g., temperature, time, amount, and concentration, including ranges, indicates approximations which may vary by (+) or (−) 10%, 5% or 1% of the disclosed values, unless specified otherwise.

As will be understood by one of skill in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member.

As utilized herein with respect to structural features (e.g., to describe shape, size, orientation, direction, relative position, etc.), the terms “approximately,” “about,” “substantially,” and similar terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g. permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic, or combinations thereof.

References herein to the positions of elements (e.g., “top.” “bottom,” “above,” “below”) are merely used to describe the orientation of various elements in the figures. It should be noted that the orientation of various elements may differ according to other exemplary embodiments, and that such variations are encompassed by the present disclosure.

Although the figures and description may illustrate a specific order of method steps, the order of such steps may differ from what is depicted and described, unless specified differently above. Also, two or more steps may be performed concurrently or with partial concurrence, unless specified differently above.

It is important to note that any element disclosed in one embodiment may be incorporated or utilized with any other embodiment disclosed herein and that various modifications may be made. For example, different surface textures may be utilized in connection with different colors for one or more portions of the autoinjector 10 according to various embodiments. It should be appreciated that various elements of certain embodiments may be incorporated or utilized with any of the other embodiments disclosed herein. All such variations are within the scope of the disclosure. Additionally, while various aspects and embodiments have been disclosed herein, other aspects and embodiments will be apparent to those skilled in the art. The various aspects and embodiments disclosed herein are for purposes of illustration and are not intended to be limiting, with the scope and spirit of the invention being indicated by the following claims.

What is claimed is:

1. A method for the acute treatment of migraine headaches with or without aura by injecting an ergoline derivative into a subject in need thereof, the method comprising:
storing the ergoline derivative in an autoinjector at room temperature in a foil pouch;
removing the autoinjector from the foil pouch;
removing a cap of the autoinjector;
pushing the autoinjector down against an injection site of the subject to depress a needle safety guard;
pressing and releasing an activation button on the autoinjector to start injection of the ergoline derivative into the subject;
holding the autoinjector down against the injection site for at least 10 seconds irrespective of whether a click is generated by the autoinjector;
confirming that the injection is complete by viewing a change of color in a viewing window of the autoinjector; and
lifting the autoinjector up and away from the injection site and allowing the safety guard to drop down and lock over a needle.

2. The method of claim 1, wherein the ergoline derivative is selected from dihydroergotamine, ergotamine, methysergide, or a pharmaceutically acceptable salt of any thereof.

3. The method of claim 1, wherein the ergoline derivative is dihydroergotamine or a pharmaceutically acceptable salt thereof.

4. The method of claim 1, further comprising generating a click from the autoinjector when the injection starts and generating a second click from the autoinjector when the injection is about to complete.

5. The method of claim 1, further comprising activating the activation button after the safety guard is depressed against the injection site.

6. The method of claim 3, wherein the pharmaceutically acceptable salt of dihydroergotamine is dihydroergotamine mesylate, and the dihydroergotamine mesylate is injected in a composition without a monoclonal antibody.

7. The method of claim 1, further comprising repeating the method (i) within a 24 hour period, (ii) within a one week period, (iii) two more times within a 24 hour period or (iv) five more times within a one week period.

8. The method of claim 1, further comprising holding the autoinjector down against the injection site for less than about 15 seconds.

9. The method of claim 3, wherein the pharmaceutically acceptable salt of dihydroergotamine is dihydroergotamine mesylate, and
wherein about 1 mg of the dihydroergotamine mesylate is injected into the subject or less than about 5 mg of the dihydroergotamine mesylate is injected into the subject.

10. The method of claim 3, wherein the pharmaceutically acceptable salt of dihydroergotamine is dihydroergotamine mesylate, and about 1 mL of a composition comprising the dihydroergotamine mesylate is injected into the subject.

11. The method of claim 3, further comprising causing an entirety of a bottommost surface of the safety guard to contact the injection site.

12. The method of claim 1, wherein pushing the autoinjector down against the injection site comprises maintaining downward force on the autoinjector throughout injection.

13. The method of claim 1, wherein the autoinjector, including the viewing window thereof, is configured to inhibit light exposure to the ergoline derivative stored in the autoinjector.

14. The method of claim 1, wherein when injection begins, a portion of the viewing window changes from a first color to a second color.

15. The method of claim 7, wherein the method is repeated within one week after the method is first performed.

16. The method of claim 5, wherein activating the activation button comprises applying less than about 20 N of force.

17. The method of claim 1, further comprising generating audible feedback from the autoinjector during the injection.

18. A method for the acute treatment of migraine headaches with or without aura by injecting dihydroergotamine mesylate into a subject in need thereof, the method comprising:
removing an autoinjector from a foil pouch, the autoinjector containing the dihydroergotamine mesylate;
removing a cap of the autoinjector;
pushing the autoinjector down against an injection site of the subject to depress a safety guard;
pressing and releasing an activation button on the autoinjector to start injection of dihydroergotamine mesylate into the subject;
holding the autoinjector down against the injection site for at least 10 seconds, wherein completeness of the injection is confirmable by a change of color viewable in a viewing window of the autoinjector; and
lifting the autoinjector up away from the injection site and allowing the safety guard to drop down and lock over a needle.

19. A method for treatment of cluster headaches by injecting an ergoline derivative into a subject in need thereof, the method comprising:
storing, in a foil pouch and at room temperature, an autoinjector containing the ergoline derivative;
removing the autoinjector from the foil pouch;
removing a cap of the autoinjector, thereby exposing a safety guard;
depressing the autoinjector against a surface of the subject to depress the safety guard;
manually actuating the autoinjector to start injection of the ergoline derivative into the subject;
maintaining the autoinjector in a position in which it is depressed against the surface for at least 10 seconds;
confirming the injection by viewing a change of color in a window of the autoinjector; and
moving the autoinjector out of contact with the surface and allowing the safety guard to move so as to be situated over a needle.

20. The method of claim 19, wherein the ergoline derivative is selected from dihydroergotamine, ergotamine, methysergide or a pharmaceutically acceptable salt of any thereof.

21. The method of claim 20, wherein the ergoline derivative is dihydroergotamine or a pharmaceutically acceptable salt thereof.

22. The method of claim 19, wherein actuating the autoinjector comprises actuating a button of the autoinjector before or after the safety guard contacts the surface of the subject.

23. The method of claim 19, further comprising holding the autoinjector down against an injection site for less than about 30 seconds.

24. The method of claim 19, further comprising holding the autoinjector down against an injection site for less than about 15 seconds.

25. The method of claim 19, wherein the autoinjector is configured to store no more than 1.5 mL of a composition comprising the ergoline derivative, and
wherein the ergoline derivative is dihydroergotamine mesylate.

26. The method of claim 19, wherein actuating the autoinjector comprises applying less than about 20 N of force.

27. The method of claim 19, further comprising generating audible feedback from the autoinjector during the injection, wherein the audible feedback is generated when injection starts and later during the injection.

28. A method for treatment of cluster headaches by injecting dihydroergotamine mesylate into a subject in need thereof, the method comprising:
removing an autoinjector containing dihydroergotamine mesylate from a foil pouch in which the autoinjector is hermetically sealed;
removing a cap of the autoinjector, thereby exposing a safety guard;
depressing the autoinjector against a surface of the subject to depress the safety guard;
manually actuating the autoinjector to start injection of dihydroergotamine mesylate into the subject;
maintaining the autoinjector in a position in which it is depressed against the surface for at least 10 seconds, wherein completeness of the injection is confirmable by viewing a change of color in a window of the autoinjector; and
moving the autoinjector out of contact with the surface and allowing the safety guard to move so as to be situated over a needle.

29. The method of claim 1, wherein an oxygen scavenger is disposed in the foil pouch.

30. The method of claim 1, wherein the autoinjector is utilizable when stored at room temperature.
